(19) United States
(12) Patent Application Publication
(10) Pub. No.: US 2012/0020830 A1
(43) Pub. Date: Jan. 26, 2012

(54) Apparatus And Method For Using Ozone As A Disinfectant

(75) Inventors: Nigel Boast, Kelowna (CA); Doug Heselton, Surrey (CA); Jim Hudson, Delta (CA); Sharma Manju, Vancouver (CA)

(73) Assignee: Viroforce Systems, Inc.

(21) Appl. No.: 13/008,862

(22) Filed: Jan. 18, 2011

Related U.S. Application Data
(63) Continuation of application No. 10/593,377, filed on Feb. 4, 2008, now abandoned.
(60) Provisional application No. 60/553,937, filed on Mar. 18, 2004.
(60) Provisional application No. 60/625,101, filed on Nov. 5, 2004.
(60) Provisional application No. 60/656,888, filed on Mar. 1, 2005.

(51) Int. Cl. A61L 2/00; A61L 2/28

(52) U.S. Cl. 422/2; 422/119

(57) ABSTRACT

A method of sterilizing a closed environment is provided in which an ozone generator is placed into the closed environment; it then generates ozone to a predetermined ozone concentration and increases the humidity of the closed environment. The ozone concentration is maintained at the predetermined ozone concentration for a predetermined period of time, and after the period of time has expired, the ozone is depleted. When the ozone concentration is reduced to a predetermined safe level, the ozone generator signals

### CROSS REFERENCE

The present application is a continuation of application Ser. No. 10/593,377 filed Feb. 4, 2008, which is the National Stage of International Application No. PCT/CA05/00412 filed Mar. 18, 2005, which claims the benefit of Provisional Application No. 60,553,937 filed Mar. 18, 2004; Provisional Application No. 60/625,101 filed Nov. 5, 2004; and Provisional Application No. 60/658,888 filed Mar. 1, 2005, the contents of which are incorporated herein in their entirety.

### FIELD OF THE INVENTION

This invention relates to tools and methods for sterilizing closed environments, and more particularly to the use of ozone to sterilize a room.

### BACKGROUND OF THE INVENTION

People traveling around the world have resulted in the rapid spread of emerging viruses and other diseases. If a disease becomes prevalent in a particular city, it can quickly spread internationally due to travel of the originating city's inhabitants. Once the disease is identified and infected individuals isolated, the disease has often already spread to high-density municipal areas, potentially in other countries.

An example of such a disease is found in the rapid spread of Severe Acute Respiratory Syndrome (SARS) which has a high morbidity and mortality rate and can be difficult to treat. It is very difficult to screen infected people and prevent them from spreading the disease. In particular, the spread of such diseases poses a high risk to the hospitality industry, and can lead to reduced earnings and share prices of public companies in the hospitality sector. The aggressive spread of SARS from Asia to other countries including the United States and Canada has challenged the airline, hospitality and tourism industries as well as hospitals. The spread of SARS also had a negative impact on affected countries' economies, including that of major cities such as Toronto.

SARS is not the only virus of concern. A variety of airborne, gastro enteric and enteric viruses, including varicella zoster (chicken pox), measles virus, rhinovirus (cold), influenza virus (flu), poliovirus, rotavirus, hepatitis A, Norwalk virus, adenovirus, and emerging viruses all represent risks of contagion and infection. The spread of bacterial infections and fungus can also be of significant concern, particularly when drug-resistant varieties occur. Such diseases are also of concern in the health care sector. For example, Clostridium difficile (a human pathogenic bacterium of the gut) is very difficult to remove when infected individuals are kept at a hospital. Health care workers and future patients may be put at risk in such situations.

Ozone has long been recognized as an effective biocide (a biochemical disinfectant), and is also a powerful deodorizer, having a number of attractive features. For example, ozone is pervasive in a closed space. Ozone is also highly effective as a viricide, and is inexpensive to administer, as ozone generators are plentiful and easy to install and operate.

Ozone is naturally formed, particularly in the upper atmosphere, when high-energy ultraviolet rays sever conventional oxygen (O₂) bonds, creating free radical oxygen atoms, which then react with other O₂ molecules to form ozone (O₃). Ozone is also formed naturally during lightning storms, at ocean beaches and waterfalls.

The structure of ozone is highly reactive, and consequently ozone has a short half-life (about 30 minutes). When ozone breaks down, it produces oxygen and a free radical oxygen atom. This oxygen free radical is a powerful oxidant.

There are several ozone generators described in the prior art. For example, U.S. Pat. No. 5,904,901 to Shimono discloses a deodorization/odor-removal/disinfection method and deodorization/odor-removal/disinfection apparatus.

Prior art relating to the sterilization of hotel rooms and the like using ozone includes JP4038957A2, which discloses a determination of the time a room should be exposed to a particular concentration of ozone. JP2237565A2 discloses an indoor sterilizing method which includes placing an ozone generator in a room, generating a level of ozone, leaving the ozone at that level for a period of time, and then decomposing the ozone.

What is missing in the prior art is a consideration of other factors besides ozone concentration and time needed to use the ozone effectively as a sterilizing agent. Also while ozone is recognized as having sterilizing properties, few tests have been carried out to determine its efficacy on new diseases.

### BRIEF SUMMARY OF THE INVENTION

A method of sterilizing a closed environment is provided, including (a) placing a ozone generator into said closed environment; (b) generating ozone to a predetermined ozone concentration; (c) increasing the humidity of said closed environment; (d) maintaining said predetermined ozone concentration for a predetermined period of time; (e) after the expiry of said period of time, depleting said ozone; and (f) when said ozone concentration is reduced to a predetermined safe level, signalling.

An ozone generator is provided including a humidifier; a timer; ozone generation means; ozone depletion means; movement means; signalling means; and detection means for detecting ozone concentration and humidity of a closed environment.

A method of inactivating a quantity of Norwalk virus in a closed environment is provided, comprising exposing the closed environment to an ozone concentration of 20 to 35 ppm for 30 to 70 minutes. It is beneficial to elevate the humidity of said closed environment while exposing the closed environment to said ozone concentration.

### BRIEF DESCRIPTION OF THE DRAWINGS

FIG. 1 is a perspective view of an ozone generator according to the invention;

FIG. 2 is a block diagram thereof; and

FIG. 3 is a flow chart showing the use of an ozone generator according to the method.

### DETAILED DESCRIPTION OF THE INVENTION

A difficulty with using ozone as a disinfectant is that the concentrations and exposure times required for ozone to be an effective disinfectant are considered to be toxic for humans. Such concentrations and exposure times may also generate noxious by-products from chemical reactions with fabrics commonly found indoors (particularly in carpets). For example, ozone may react with chemicals in carpets to create formic acid. Exposure to elevated ozone concentrations may irritate the lungs and have other side effects, including throat irritation, shortness of breath and coughing. Consequently several agencies have discouraged the use of ozone to sanitize indoor spaces and have set maximum safe levels of ozone to be from 0.05 parts per million (“ppm”) to 0.10 ppm for an eight hour exposure. Unless otherwise stated to references to ozone in this document refer to ozone in a gaseous state.

Ozone is effective against many types of organisms, including retroviruses, both enveloped and naked viruses, bacteria and fungus. Specific diseases which ozone has been shown to be effective against include: MS2 Coliphage; Poliovirus Type 1 and Type 3; Hepatitis A; Enteroviruses; Rotaviruses; HIV; SA11 and enteric viruses; Influenza viruses; the Norwalk virus and Rhinoviruses. Ozone may also be used to kill SARS viruses, infectious prions, and bacteria, and can also decontaminate foodstuffs and sterilize medical equipment. For further information about the general efficacy of ozone as a viricide see Appendix A entitled “Ozone: A Viricidal Agent for Conventional and Emerging Viruses”.

The level of ozone concentration required to be effective and achieve over 95% (and often over 99%) morbidity rates of viruses and other disease causing agents varies depending on the time the agents are exposed to the ozone. One constant is that the ozone concentration is well above the safe levels for human exposure and therefore precautions should be taken to prevent such exposure. Ozone concentrations of approximately 100 ppm are very effective to kill infectious agents and may require exposure times for as little as 10 to 15 minutes. Lower ozone concentrations (for example as low as 20 ppm to 25 ppm) are also effective, although, in the case of such lower quantities of ozone, it takes more time (such as 20 to 30 minutes) for the ozone to be effective.

### The Process of Using Ozone as a Disinfectant

The present invention includes portable equipment, specifications and operating procedures to provide adequate ozone exposure in indoor spaces to achieve an effective degree of sanitization followed by rapid removal of the ozone and attendant gaseous by-products from the reaction of ozone with carpet and furniture fabrics.

The invention includes identifying the variables impacting the safe and effective use of ozone as a disinfectant in the hospitality and other industries. In summary, the invention provides for:

1. Rapid elevation of ozone levels within a fixed interior space, combined with suitatue humidity control and turbulent airflow;

2. Measurement and control of effective exposure to sanitizing ozone optimum for the use of ozone as a viricide for use on various surfaces commonly found in the fixed space (e.g. in a hotel room); and

3. Rapid consumption of ozone and gaseous aldehyde by-products to reduce their concentrations to levels deemed safe for human exposure.

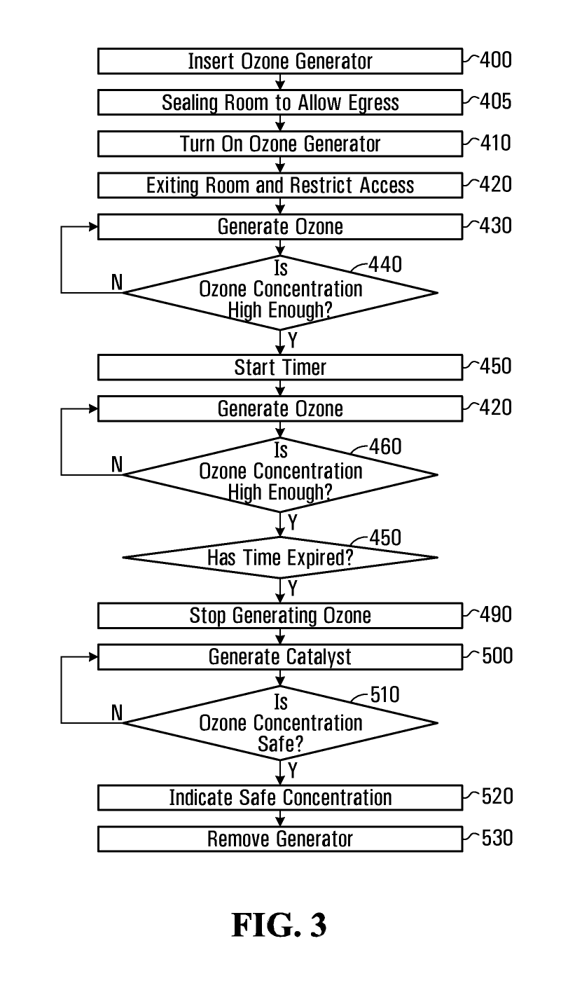

As an example, as seen in FIG. 3, a preferred method according to the invention may include the following steps:

a) inserting a portable ozone generator in a closed environment, such as a hotel room (step 400);

b) elevating and maintaining the ozone concentration in the closed environment to a level sufficient to act as a disinfectant and viricide taking into account the humidity, size, temperature and airflow of the closed environment (step 410);

c) restricting access to the closed environment while the ozone levels are elevated to prevent human exposure while the ozone concentration is dangerously high (step 420);

d) consuming the ozone and any gaseous aldehyde by-products (possibly including the use of a catalyst) for a period of time taking into account the ozone levels, the humidity, the temperature, the airflow and the size of the closed environment, until the ozone concentration is below toxic levels (step 500); and

e) removing the portable ozone generator from the closed environment (step 530).

In further detail, with reference to FIG. 3, the process begins with the insertion of an ozone generator into a closed interior room (step 400). Examples of appropriate rooms include hotel rooms, cruise ship cabins, hospital rooms and airplane cabins. The room is preferably easily cut off from public access (step 405) so that employees or guests will not be exposed to high concentrations of ozone. Examples of closing a room include simply locking the door of a hotel room or cruise ship cabin when it is not in use by a guest. Windows should be closed and any ventilation systems turned off. Note that as the user is still inside the room, it is important that it not be difficult to exit the closed environment quickly.

The user will then preferably turn on the ozone generator (step 410) and exit the closed environment (step 420). Preferably the ozone generator has a timer such that when it is turned on, there is a period of time (for example one or two minutes) before the ozone generator will begin generating ozone. This provides time for the user to exit the closed environment without exposure to the ozone.

In some embodiments of the invention, the user will have to adjust the ozone generator so that it will produce the appropriate amount of ozone within the appropriate time based on humidity, temperature, air flow and the like. It may also be necessary for the user to enter information about the room size (for example a menu of options such as “Suite”, “Single” or “Double” could be displayed from which the appropriate selection is made). Alternatively, in a preferred embodiment, the ozone generator will measure these indicia, like temperature and humidity and automatically calculate the appropriate concentration of ozone and time that it should be maintained.

The next step is to restrict access to the closed environment (step 420) while the ozone concentration is elevated to prevent exposure to the ozone. The closed environment does not need to be airtight, for example closing the doors and windows of a hotel room is sufficient. Fans within the room should be turned off. The entrance to the closed room should be locked and possibly a sign or warning light used to indicate that entry should not be permitted during the period when ozone concentrations are elevated.

The ozone generator may also be able to adjust certain factors of the closed environment in order that the ozone will more efficiently act as a viricide. For example the ozone generator may also have the ability to increase the humidity of the closed environment, which make the ozone more efficient as a viricide. This in turn may allow the ozone generation and ozone concentration maintenance periods to be shorter.

The ozone generator then generates ozone (step 430) until the appropriate concentration is reached (step 440). This concentration is maintained (step 440) for the specified period of time (steps 460 and 470). Examples of sufficient ozone concentrations in a typical hotel room or cruise ship cabin would be 40 to 50 ppm for about 10 to 15 minutes or a concentration between 20 and 35 ppm for about 20 to 35 minutes. Increased humidity levels can shorten the time needed.

After the ozone concentration has reached the desired level and has been maintained at that level for a sufficient period of time (step 480), the ozone generator stops generating ozone (step 490). The ozone then begins to dissipate, both naturally, and preferably by the generation of an appropriate catalyst (step 500). The ozone concentration is measured (step 510) as the ozone is dissipated (as are the gaseous aldehyde by-products) for a period of time taking into account the ozone levels, the humidity, the temperature, the airflow and the size of the closed environment, until the ozone concentration is below toxic levels at which point the ozone generator signals the room is safe to enter using an LED, a noise or the like (step 520).

Once the appropriate amount of time has passed and the ozone generator has indicated the ozone concentration is sufficiently low, the ozone generator is removed from the closed environment and can be used in another closed environment (step 530).

### The Ozone Generator

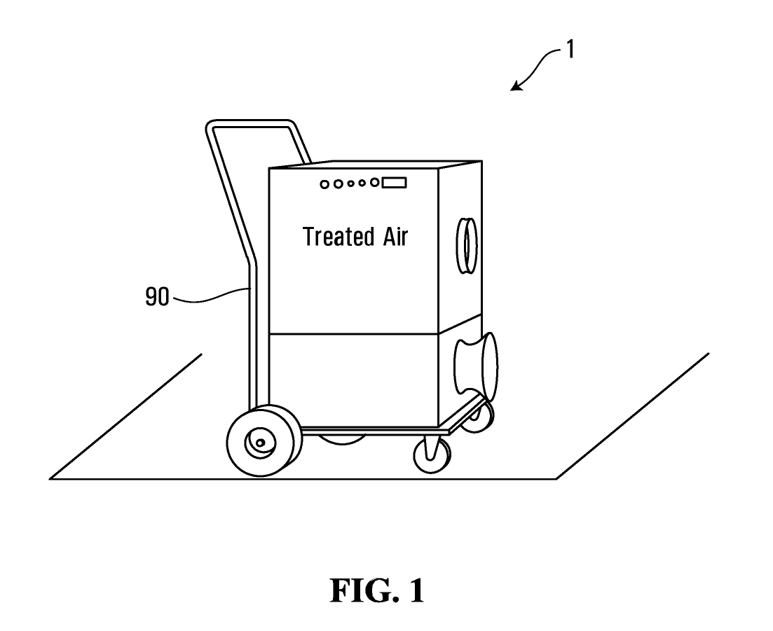

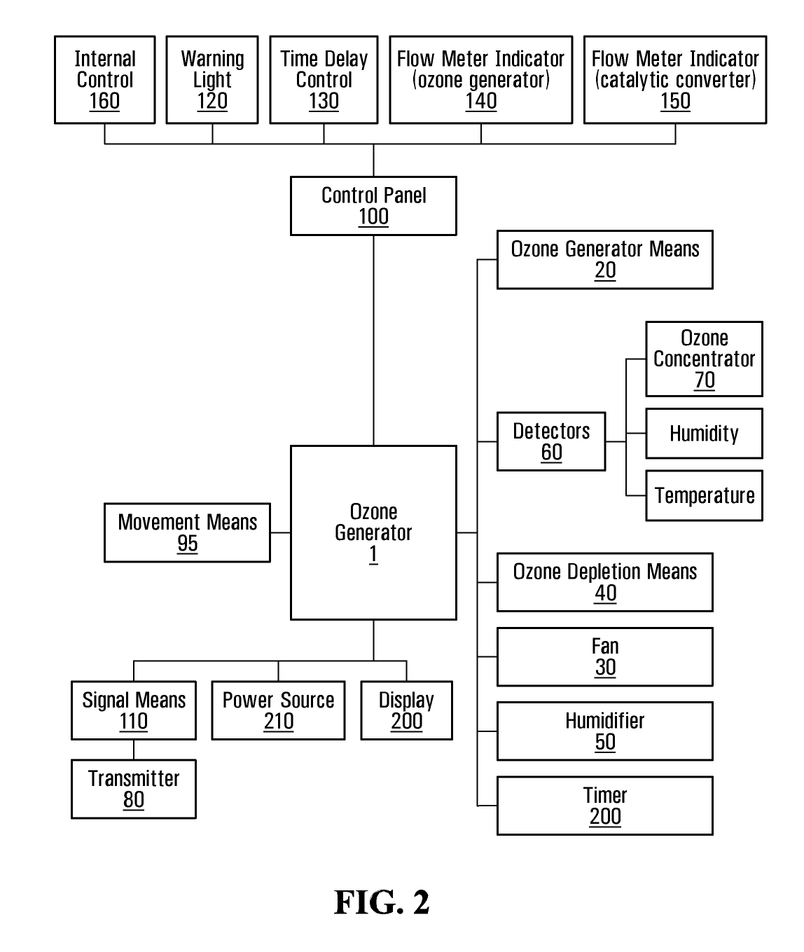

The previously described method can be used with a variety of zone generators, however a preferred ozone generator is shown in FIGS. 1 and 2. The ozone generator, generally indicated as 1, preferably generates gaseous ozone using corona discharge or ultra violet light or other ozone generation means as known in the art. The corona discharge process can create ozone using air in the closed environment passed through ozone generator 1 by fan 30, or alternatively air can be introduced into the closed space or industrial or medical oxygen. The ozone generator preferably also has an ozone depletion means 40 such as an ozone scrubber or catalytic converter, and a humidifier 50. Also the generator preferably has detectors 60, particularly a detector for the concentration of ozone 70 in the closed environment.

The ozone scrubber or catalytic converter (also referred to as ozone depletion means) allows the ozone generator 1 to quickly deplete the concentration of ozone to levels which are acceptable for human habitation. A catalytic converter uses substances such as manganese dioxide, treated or activated carbon, or a combination of both. A catalytic converter will also deplete the ozonated air of aldehyde, nitroxides and any other noxious gases generated as by products of the ozone reacting with articles in the environment, such as carpets. Activated carbon can be used to reduce the levels of noxious by-products caused by the ozone reactions with carpet and the like. Another factor in the depletion of the ozone, is the natural half-life of ozone, which is about 25 to 30 minutes.

The ozone generator 1 also preferably has a humidifier 50. The humidifier 50 is used to modify the relative humidity of the air volume in conjunction with the other operations of the generator. Accordingly, the humidifier may be used before, during and/or after the ozone generation process as necessary. As higher levels of humidity tend to make the ozone more effective as a viricide, in most closed environments the humidifier 50 will be engaged to increase the humidity of the closed environment.

The ozone generator 1 should either be sufficiently small and light enough to be easily carried or should be mounted on a trolley 90 (as seen in FIG. 1) or affixed with other movement means 95, such as wheels. Alternatively the ozone generator 1 could be a fixture with the closed environment. In a preferred embodiment the generator is affixed to an ergonomically suitable trolly 95 so that it can easily be moved from room to room within a larger structure (such as a hotel).

The ozone generator 1 also preferably has detectors 60 means to detect the ozone levels 70 within the closed environment. This is used so that users can determine when the ozone concentration is low enough to allow safe entry into a room. In a preferred embodiment of the invention the generator will indicate that the ozone concentration is safe and transmit a signal using transmitter 80 to a device (a mobile phone, PDA or the like) indicating that the room is now safe to enter. Alternatively the signal can be sent to a control panel 100 which will manipulate a LED on the outside of the room (e.g. red for high concentrations, and green for lower safe concentrations).

In yet another alternative embodiment, the generator has an LED or similar signal means 110 such that a user entering the closed environment will be immediately aware that the ozone levels are still too high for safety and can exit the closed environment.

The ozone generator also preferably has one or more of the following components:

1. a timer 110 to record the number of hours or minutes the generator has been operating and to turn off the generator when the appropriate time has passed;

2. a warning light 120 to indicate that the ozone generator is generating ozone;

3. a time delay switch 130 to allow for a delay before the ozone generator begins to generate ozone, allowing the user to exit the closed environment;

4. one or more other time delay switches for the operation of the scrubber, humidifier, and other features;

5. a flow meter 140 to indicate that there is an air flow moving through the ozone generator;

6. a flow meter 150 to indicate that there is an air flow moving through the catalytic converter;

7. an instrument panel to indicate which part of the apparatus is working either individually or with others;

8. further alarms included in the instrumentation that would indicate a malfunction of the generator;

9. an internal control 160 to allow for variance of the ozone concentration to be achieved;

10. sliding inspection panels to allow for easy maintenance and inspection of the apparatus; and

11. separate electric fittings and plugs to allow for ancillary apparatus such as an additional scrubber to be connected to the apparatus.

Ozone generator 1 also has power source 210 which can be a plug for insertion into a suitable outlet, or batteries. Ozone generator also has displays 200 preferably showing the current ozone concentration, humidity and temperature.

### Use Example 1

### Hotels

Hotels are used to frequent visitors in a particular room, often only staying a single night. Hotels are also one of the worst effected by disease scares as in the case of SARS, as tourism is one the industries most keenly effected. Hotels have also been using ozone at low concentrations to reduce odours in rooms.

As used in hotels according to the method, a maid after initially cleaning a vacated room (preferably after the guest had checked out) would place the ozone generator in the room set it for the specified ozone concentration and time, and leave the room (and locking the door), returning when the time had passed and the ozone concentration was reduced to safe levels. The ozone generator can then be taken to the next appropriate room.

At the end of the process, the ozone would kill the viruses, bacteria and fungi left by the departing persons).

### Use Example 2

### Airplanes

The airline industry is another industry prone to losses when fear of a disease outbreak strikes. To use the method according to the invention on an airliner, after the airliner is initially cleaned, one or more ozone generators should be turned on and the selected ozone concentration maintained for a period of time. During this time access to the interior of the airplane should be prevented.

Once the necessary time has passed, and the ozone concentrations are safe, the interior of the airplane is access and the ozone generators can be removed.

### Use Example 3

### Cruise Ships

Cruise ships present an environment where a disease can spread rapidly due to the confinement of a large number of people in a small environment. The method according to the invention is useful when the ship is docked and few are about, in which case it is used in a manner very similar to that of the hotel example described previously. Alternatively, the ozone generator could be used within a room when the inhabitants report certain symptoms.

### Use Example 4

### Hospitals

A yet further example of a location in which to use the method according to the invention is a hospital. Obviously hospitals are areas in which viruses, bacteria and other disease causing agents are common as those diseased may end up in such a location. When a hospital room is vacated, perhaps even only temporarily, the method according to the invention could be carried out to kill any viruses or bacteria left by the last patient staying in such rooms. It may be beneficial to use the ozone generator in emergency areas and the like when such area is exposed to a particularly problematic disease (such as SARS).

### Effectiveness of Ozone

Generally tests were conducted to show that ozone gas can efficiently inactivate (kill) five selected viruses tested, namely, herpes simplex virus, influenza virus, corona virus, poliovirus and rhinovirus. These viruses were found to be vulnerable to ozone in a gaseous state on surfaces such as glass, plastic, steel, wood and fabric. Increasing the concentration of ozone and greater times of exposure were more effective, as anticipated, and increasing the relative humidity also significantly increased the antiviral efficacy.

### Experiment #1

Ozone was generated within a chamber to provide an ozone concentration of approximately 100 ppm for 30 minutes on a variety of surfaces, including glass slides, steel disks, etc.. Relative humidity and temperature were recorded.

Herpes Simplex Virus (“HSV”), Feline calicivirus (“FCV”), and Mulluscum Contagiosum Virus (“MCV”) were all dramatically inactivated by exposure to ozone gas. Typically a dosage of 100 ppm for 20-30 minutes reduced the virus by more than 99%. Shorter exposure times resulted in significant though smaller reductions. Thus 10 minutes inactivated approximately 90-95% virus infectivity, whereas shorter time periods were less effective. It appeared, from a number of the time course studies made, that a period of between 5 and 10 minutes exposure to ozone was required to absorb the gas and effect the appropriate chemical processes, before loss of infectivity occurred. Poliovirus was also inactivated by ozone under similar conditions.

Exposure of the viruses to ozone was made on samples dried on six different surfaces, relevant to materials encountered in the hospitality industry, glass, plastic, stainless steel, wood, fabric, and carpet. Several viruses were evaluated on each surface, though not every permutation was feasible because of time constraints and cost. In general, the viruses were susceptible to ozone on glass, plastic, steel, wood, and fabric.

The results of numerous time course experiments, with different virus-surface combinations, confirmed that increasing time of exposure resulted in greater inactivation of virus, and in some cases no virus infectivity could be detected at all after 30 minutes exposure.

In several experiments the effect of relative humidity was examined by incorporating a container of warm water into the chamber during exposure. It was difficult to control exact humidity levels in this manner; nevertheless it was clear that in high humidity virus was inactivated by ozone much more efficiently than in ambient humidity (which was usually 45-50%).

### Experiment #2

A further experiment was conducted to test the effect of ozone gas against selected viruses, under conditions similar to those in a hotel room. The aim was to measure the amount of ozone inactivation of HSV in several different locations within a test room and to compare the efficacy of ozone inactivation of three different viruses (HSV, poliovirus and rhinovirus) placed within the test room.

The three samples of HSV were inactivated (killed) by 98%, 99.4% and 97.8%. The ozone concentration was 28 ppm and the time of exposure was 60 minutes (it also took 30 minutes to reach that ozone concentration from a starting point of 0).

As the inactivation was similar at three different locations within the room indicating that the ozone gas should be very effective at inactivating viruses within a large room.

### Experiment #3

A further experiment was conducted to evaluate the effect of ozone gas against FCV, the surrogate virus for Norwalk virus, in comparison with HSV and poliovirus, under conditions of reduced ozone doses and high humidity.

The FCV was inactivated by 99.91%; the poliovirus was inactivated by much more than 99.6%; and the HSV was inactivated by much more than 99%. The closed environment used for these tests, was provided an atmosphere of high humidity, and with substantially reduced ozone dosage (between 20 ppm and 40 ppm) for about 15 minutes. It was concluded that FCV can be inactivated more than 99.9% by exposure to ozone gas in the presence of high relative humidity and it should be possible to inactivate this virus (and by extrapolation Norwalk virus) even further by optimizing the ozone dosage and humidity.

### Experiment #4

A further experiment was conducted to develop an appropriate and relevant experimental system for testing the efficacy of quantified ozone doses in inactivating (i.e. killing) known amounts of several important human viruses; to derive viricidal killing curves for known doses of ozone gas against samples of dried viruses on several different surfaces relevant to the hospitality industry; to compare the viricidal efficacy of ozone gas against five selected viruses known to be important in human health; to examine the effects of different parameters on the viricidal efficacy of ozone gas, including: concentration of ozone, time of exposure, and relative humidity; and to consider the potential for additional applications of ozone gas as a sterilizing agent in other situations where viral and microbial agents could pose threats.

The experiments showed that ozone gas can efficiently inactivate (kill) all of the five selected viruses tested, namely, herpes simplex virus, influenza virus, corona virus, rhinovirus, and poliovirus. These viruses are vulnerable to ozone gas in the dried state on different surfaces, such as glass, plastic, steel, wood and fabric. Increasing doses of ozone and greater times of exposure were more effective, as anticipated, and increasing relative humidity also significantly increased the antiviral efficacy.

Based on these results we conclude that the viruses tested are efficiently inactivated by gaseous ozone, on each of the surfaces tested, under conditions relevant to practical applications. Therefore ozone gas also has potential as a safe antiviral and anti-microbial agent in various other situations that are accessible to a small, portable, ozone generating machine.

HSV, FV, and MCV were all dramatically inactivated by exposure to ozone gas. Typically a dosage of 100 ppm for 20 to 30 minutes reduced the virus by more than 99%. Shorter exposure times resulted in significant though smaller reductions. Thus 10 minutes inactivated approximately 90-95% of the virus infectivity, whereas shorter time periods were less effective. It appeared, from a number of the time course studies made, that a period of between 5 and 10 minutes exposure to ozone was required to absorb the gas and effect the appropriate chemical processes, before loss of infectivity occurred. Presumably oxidation of particular viral components is required, and that this process requires several minutes. Following this process, inactivation, i.e. loss of infectivity, is rapid.

Exposure of the viruses to ozone was made on samples dried on six different surfaces, relevant to materials encountered in the hospitality industry, glass, plastic, stainless steel, wood, fabric, and carpet. Several viruses were evaluated on each surface. In general, the viruses were susceptible to ozone on such surfaces.

In several experiments the effect of relative humidity was examined by incorporating a container of warm water into the chamber during exposure. It was difficult to control exact humidity levels in this manner; nevertheless it was clear that in high humidity the virus was inactivated by ozone much more efficiently than in ambient humidity (which was usually 45-50%).

### Experiment #5

Further experiments were conducted to determine the inactivation of the Norwalk virus and to do research regarding an ozone scrubber. It had already been demonstrated that several viruses, including the feline calicivirus (the recommended surrogate virus for testing Norwalk virus susceptibility to anti-viral agents), could be inactivated by ozone gas.

The objective of the experiment was to optimize the ozonation protocols in order to minimize the effective dose and exposure times required, to determine the degree of relative humidity preferred, and to confirm the optimal protocols for virus specimens resembling field conditions (i.e. in different biological fluids and on “unclean surfaces”).

The feline calicivirus is used in these test procedures because Norwalk virus itself cannot be grown and measured in cell cultures. However, once optimal conditions for ozone inactivation of calicivirus have been determined, then reference stool specimens known to contain Norwalk virus can be tested.

The data confirmed that FCV, and therefore Norwalk virus, can be efficiently inactivated by our ozone generator under standard conditions and at durations, temperature and humidity levels which would be appropriate for the cruise liner and hotel industries.

Other disease causing agents such as viruses and bacteria that ozone is effective against include: Clostridium difficile (a human pathogenic bacterium of the gut); Antibiotic-Resistant bacteria (E. coli, Staphylococcus and Streptococcus, including the multiple antibiotic -resistant strain (MRSA) of Staph); Candida albicans (a yeast); and fungi growing on different surfaces.

Although the particular preferred embodiments of the invention have been disclosed in detail for illustrative purposes, it will be recognized that variations or modifications of the disclosed apparatus lie within the scope of the present invention.

What is claimed is:

1. A method of sterilizing a closed environment comprising:
(a) inserting a portable ozone generator within the closed environment;
(b) selecting a room size from a menu of standard options;
(c) activating, by a user, a timer on said portable ozone generator;
(d) restricting access to said closed environment to prevent public access;
(e) after expiry of said timer, generating gaseous ozone into said closed environment to a predetermined ozone concentration between 20 and 50 ppm;
(f) increasing the humidity of said closed environment;
(g) maintaining said predetermined ozone concentration for a predetermined period of time between 10 and 35 minutes;
(h) after the expiry of said period of time, depleting said ozone using a catalytic converter; and
(i) when said ozone concentration is reduced to a maximum level of 0.10 part per million, signalling.

2. The method of claim 1 further comprising the step:
(j) allowing access to said closed environment.

3. The method of claim 1 wherein said portable ozone generator is provided with wheels, and is inserted into said closed environment using said wheels.

4. The method of claim 3 wherein said catalytic converter uses manganese dioxide.

5. The method of claim 3 wherein said catalytic converter uses activated carbon.

6. The method of claim 4 wherein said ozone level is maintained between 40 and 50 ppm for between 10 and 15 minutes.

7. The method of claim 4 wherein said ozone level is maintained between 20 and 35 ppm for between 20 to 35 minutes.

8. The method of claim 7 wherein in step (i) said signalling is done with a noise.

9. The method of claim 7 wherein in step (i) said signalling is done with a LED.

10. The method of claim 8 wherein said closed environment is a hotel room.

11. The method of claim 8 wherein said closed environment is a cruise ship cabin.

12. The method of claim 10 wherein during step (a) ventilation systems within said environment are turned off

13. A sterilizer comprising:
a humidifier;
gaseous ozone generation means;
ozone depletion means;
movement means;
signal means, said signal means capable of indicating a level of ozone of 0.10 part per million or less;
detectors for detecting ozone concentration and humidity of the closed environment; and
a timer, said timer, once selected by a user, configured to actuate said gaseous ozone generation means after a delay thereby allowing said user to leave a closed environment.

14. The apparatus of claim 13 wherein said ozone depletion means uses manganese dioxide.

15. The apparatus of claim 14 wherein said sterilizer includes a handle.

16. The apparatus of claim 15 wherein said signal means emits a noise.

17. The apparatus of claim 15 wherein said signal means is a LED.

18. The apparatus of claim 17 wherein said ozone depletion means also uses activated carbon.
